(19) United States
(12) Patent Application Publication
(10) Pub. No.: US 2010/0029931 A1
(43) Pub. Date: Feb. 4, 2010

(54) METHOD FOR PRODUCING LACTAM COMPOUND

(76) Inventors: Akihiro Shibamoto, Hyogo (JP); Takahiro Iwahama, Hyogo (JP); Tatsuya Nakano, Hyogo (JP)

Correspondence Address: BIRCH STEWART KOLASCH & BIRCH, PO BOX 747, FALLS CHURCH, VA, 22040-0747, US

(21) Appl. No.: 12/519,662

(22) PCT Filed: Dec. 20, 2007
(86) PCT No.: PCT/JP2007/074500

§ 371 Date: Jun. 17, 2009

(30) Foreign Application Priority Data
Dec. 22, 2006 (JP) 2006-346628

(51) Int. Cl. C07D 201/04

(52) U.S. Cl. 540/464

(57) ABSTRACT

Disclosed is a method for industrially efficiently producing a lactam compound having 8 to 15 carbon atoms at low cost by allowing a rearrangement reaction of a cyclic oxime compound to proceed without causing large amounts of by-products such as ammonium sulfate.

[Solving Means] Disclosed is a method for producing a lactam compound, which includes the step of rearranging a cyclic oxime compound in a nonpolar solvent B in the presence of an aromatic compound A to give the lactam compound, in which the aromatic compound A has a leaving group bonded to a carbon atom constituting its aromatic ring and contains, as an atom constituting the aromatic ring, a heteroatom, or a carbon atom bonded with an electron-withdrawing group, the cyclic oxime compound is represented by following Formula (1):

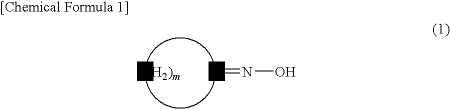

wherein “m” denotes an integer of 7 to 14,
and the lactam compound is represented by following Formula (2):

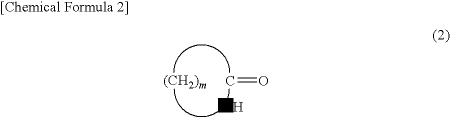

wherein “m” is as defined above.

### Technical Field

The present invention relates to methods for producing lactam compounds having 8 to 15 carbon atoms, which lactam compounds are useful typically as raw materials for pharmaceutical drugs (medicines), agricultural chemicals, dyestuffs, and polyamides. More specifically, it relates to methods for producing corresponding lactam compounds from cyclic oxime compounds having 8 to 15 carbon atoms through a rearrangement reaction.

### BACKGROUND ART

Techniques for producing corresponding lactams from raw material cyclic oxime compounds through “Beckmann rearrangement” are industrially very important. The production of these compounds has been conducted by a process of acting fuming sulfuric acid in a stoichiometric amount or more, but this process raises an issue of by-production of large amounts of ammonium sulfate to be treated.

To avoid this problem, there has been proposed a process of carrying out a reaction in a polar solvent by using a specific aromatic compound as a Beckmann rearrangement catalyst that places less load on the environment, in which the aromatic compound contains at least one carbon atom bonded with a leaving group as an atom constituting its aromatic ring, contains as a total of at least three atoms selected from heteroatoms and carbon atoms having an electron-withdrawing group as atoms constituting the aromatic ring, and in which two of the three atoms selected from heteroatoms or carbon atoms each having an electron-withdrawing groups are positioned each at the ortho position or para position of the carbon atom bonded with a leaving group (see Non-patent Document 1 and Patent Document 1). This process, however, uses a polar solvent in the reaction, and the polar solvent should be removed before separation of a reaction product lactam compound from the catalyst through an extraction operation using an organic solvent and water, because the polar solvent will inhibit the separation. This process is therefore disadvantageous in respect of energy and process from the viewpoint as a process for the industrial production of lactam compounds.

Non-patent Document 1: J. AM, CHEM. SOC. 2005, 127, 11240-11241

Patent Document 1: Japanese Unexamined Patent Application Publication (JP-A) No. 2006-219470

### DISCLOSURE OF INVENTION

### Problems to be Solved by the Invention

Accordingly, an object of the present invention is to provide a method for industrially efficiently producing a lactam compound having 8 to 15 carbon atoms at low cost, by allowing a rearrangement reaction of a cyclic oxime compound to proceed without causing large amounts of by-products such as ammonium sulfate.

Another object of the present invention is to provide a method for producing a lactam compound having 8 to 15 carbon atoms, according to which the separation of a reaction product and a catalyst after the completion of the reaction can be conducted in a simplified manner.

### Means for Solving the Problems

To achieve the above objects, the present inventors made intensive investigations on methods for producing corresponding lactam compounds from cyclic oxime compounds by using, as a catalyst, an aromatic compound containing a leaving group bonded to a carbon atom constituting its aromatic ring and containing, as an atom constituting the aromatic ring, a heteroatom, or a carbon atom having an electron-withdrawing group bonded thereto. As a result, they have found that, a reaction for producing a lactam compound from a cyclic oxime compound having a small ring with about 5 or 6 carbon atoms does not substantially proceed in a nonpolar solvent; but, in contrast to this, a reaction for producing a corresponding lactam compound from a cyclic oxime compound having 8 to 15 carbon atoms unexpectedly very smoothly proceeds in such a nonpolar solvent, and whereby the reaction product lactam compound after the completion of the reaction can be separated from the used catalyst in a simple manner of merely adding water to the reaction mixture and carrying out extraction. The present invention has been made based on these findings.

Specifically, the present invention provides a method for producing a lactam compound, which method includes the step of carrying out rearrangement of a cyclic oxime compound in a nonpolar solvent B in the presence of an aromatic compound A to give the lactam compound, in which the aromatic compound A has a leaving group bonded to a carbon atom constituting its aromatic ring, the aromatic compound A contains, as an atom constituting its aromatic ring, a heteroatom, or a carbon atom bonded with an electron-withdrawing group, the cyclic oxime compound is represented by following Formula (1):

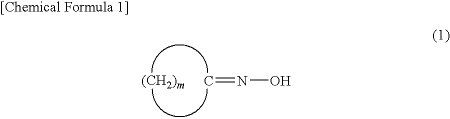

wherein “m” denotes an integer of from 7 to 14,
and the lactam compound is represented by following Formula (2):

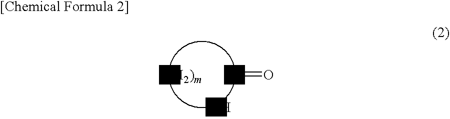

wherein “m” is as defined above.

The aromatic compound A includes an aromatic compound containing, as a constituent of the aromatic ring, a structure represented by following Formula (3):

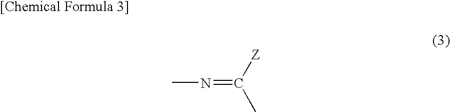

wherein Z represents a halogen atom or an —OR group, where R represents an organic group.

Z in Formula (3) includes chlorine atom. Examples of R in Formula (3) include a fluorine-containing branched-chain aliphatic group represented by following Formula (4):

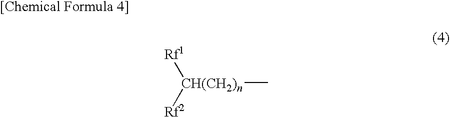

wherein Rf¹ and Rf² are the same as or different from each other and each represent a perfluoroalkyl group having 1 to 8 carbon atoms; and “n” denotes an integer of from 0 to 8; and a group corresponding to the cyclic oxime compound represented by Formula (1) and being represented by following Formula (5):

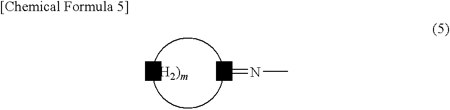

wherein “m” denotes an integer of from 7 to 14.

The nonpolar solvent B is preferably a hydrocarbon. The nonpolar solvent B can be, for example, a cycloalkane corresponding to the cyclic oxime compound represented by Formula (1) In the reaction, an acid may be used as a promoter.

### Advantages

According to the present invention, lactam compounds having 8 to 15 carbon atoms can be produced in a high yield, because rearrangement reactions of cyclic oximes can be carried out without causing large amounts of by-products such as ammonium sulfate, and whereby problems occurring in known methods for producing lactam compounds, such as removal and disposal of such by-products, can be avoided. Additionally, the separation between reaction products and the used catalyst after the completion of the reaction can be carried out in a simple manner typically of an extraction operation using water. Subsequently, lactam compounds having 8 to 15 carbon atoms can be industrially efficiently produced at low cost.

### BEST MODES FOR CARRYING OUT THE INVENTION

### Aromatic Compound A

According to the present invention, an aromatic compound A is used as a catalyst, which aromatic compound A has a leaving group bonded to a carbon atom constituting its aromatic ring and contains, as an atom constituting its aromatic ring, a heteroatom, or a carbon atom bonded with an electron-withdrawing group.

Examples of the aromatic ring include aromatic hydrocarbon rings and aromatic heterocyclic rings. Exemplary aromatic hydrocarbon rings include monocyclic aromatic hydrocarbon rings such as benzene ring; and polycyclic aromatic hydrocarbon rings including fused rings such as naphthalene ring, anthracene ring, fluorene ring, and phenanthrene ring, as well as biphenyl ring, and terphenyl ring. Exemplary aromatic heterocyclic rings include five-membered aromatic heterocyclic rings such as pyrrole ring, furan ring, thiophene ring, imidazole ring, pyrazole ring, triazole ring, tetrazole ring, oxazole ring, isoxazole ring, and thiazole ring; six-membered aromatic heterocyclic rings such as pyridine ring, pyrazine ring, pyrimidine ring, pyridazine ring, and triazine ring; and fused heterocyclic rings such as indole ring, benzimidazole ring, benzotriazole ring, quinoline ring, bipyridyl ring, and phenanthroline ring. Of the aromatic hydrocarbon rings, benzene ring is especially preferred. Of the aromatic heterocyclic rings, nitrogen-containing heterocyclic rings such as pyridine ring and triazine ring are preferred.

The leaving group bonded to a carbon atom constituting the aromatic ring is not specifically limited, as long as being a group capable of leaving, and examples thereof include halogen atoms (fluorine atom, chlorine atom, bromine atom, and iodine atom), diazonium group, sulfonyl halide groups (e.g., sulfonyl chloride group), carbonyl halide groups (e.g., carbonyl chloride group), and —OR groups wherein R represents an organic group. Exemplary organic groups as R include sulfonyl groups (e.g., arylsulfonyl groups such as benzenesulfonyl group, p-toluenesulfonyl group, and naphthalenesulfonyl group; and alkanesulfonyl groups such as methanesulfonyl group, trifluoromethanesulfonyl group, and ethanesulonyl group), haloalkyl groups [e.g., haloalkyl groups having about 1 to about 17 carbon atoms including fluorinated alkyl groups such as difluoromethyl group, trifluoromethyl group, tetrafluoroethyl group, pentafluoroethyl group, and fluorine-containing branched-chain aliphatic groups represented by Formula (4); and chlorinated alkyl groups such as trichloromethyl group, of which haloalkyl groups having about 1 to about 10 carbon atoms are preferred], alkylideneamino groups, cycloalkylideneamino groups [e.g., groups represented by Formula (5) and corresponding to the cyclic oxime compounds represented by Formula (1)].

In the fluorine-containing branched-chain aliphatic groups represented by Formula (4), Rf¹ and Rf² are the same as or different from each other and each represent a perfluoroalkyl group having 1 to 8 carbon atoms; and “n” denotes an integer of from 0 to 8. Exemplary perfluoroalkyl groups having 1 to 8 carbon atoms include trifluoromethyl group, pentafluoroethyl group, and heptafluoropropyl group. Representative examples of the fluorine-containing branched-chain aliphatic groups represented by Formula (4) include hexafluoroisopropyl group.

Representative examples of the groups represented by Formula (5) and corresponding to the cyclic oxime compounds represented by Formula (1) include cyclooctylideneamino group, cyclodecylideneamino group, cyclododecylideneamino group, and cyclopentadecylideneamino group.

Exemplary heteroatoms as an atom constituting the aromatic ring include nitrogen atom, oxygen atom, sulfur atom, and silicon atom. Among them, nitrogen atom is preferred. Though not especially limited, examples of an electron-withdrawing group, if contained as being bonded to a carbon atom constituting the aromatic ring, include cyano group; halomethyl groups such as trifluoromethyl group and trichloromethyl group; nitro group; carbonyl halide groups; acyl groups; and sulfonyl groups. The aromatic compound A preferably contains, as atoms constituting the aromatic ring, three or more atoms selected from heteroatoms, and carbon atoms that are bonded with an electron-withdrawing group. Additionally, two of the atoms selected from heteroatoms, and carbon atoms that are bonded with an electron-withdrawing group are preferably positioned at the ortho position or para position of the carbon atom bonded with the leaving group.

Preferred exemplary aromatic compounds A include aromatic compounds containing a structure represented by Formula (3) as a constituent of the aromatic ring. In Formula (3), Z represents a halogen atom or an —OR group, where R represents an organic group. Exemplary halogen atoms as Z include fluorine atom, chlorine atom, bromine atom, and iodine atom. Among them, chlorine atom is preferred. Exemplary organic groups as R are as above.

Exemplary aromatic compounds each containing a structure represented by Formula (3) as a constituent of the aromatic ring include triazine derivatives represented by following Formula (3a), pyrazine derivatives represented by Formula (3b), pyrimidine derivatives represented by Formula (3c), pyridazine derivatives represented by Formula (3d), and pyridine derivatives represented by Formula (3e):

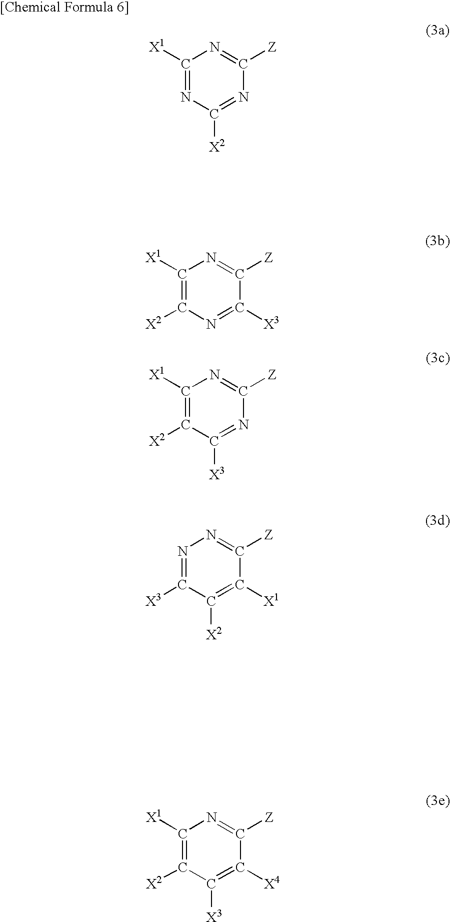

wherein Z represents a halogen atom or an —OR group, where R represents an organic group; X¹, X², X³, and X⁴ are the same as or different from one another and each represent a hydrogen atom, a halogen atom, an alkyl group, a haloalkyl group (e.g., trifluoromethyl group, difluoromethyl group, or trichloromethyl group), an aryl group, a cycloalkyl group, a hydroxyl group, an alkoxy group, an aryloxy group, a haloalkoxy group, a mercapto group, a carboxyl group, a substituted oxycarbonyl group, a formyl group, an acyl group, an acyloxy group, a nitro group, a sulfo group, a cyano group, an amino group, an oxyamino group, or another organic group, where at least two of X¹, X², X³, and X⁴ may be combined to form an aromatic or non-aromatic ring together with an atom constituting the ring in each formula.

Exemplary haloalkoxy groups as X¹, X², X³, and X⁴ include haloalkoxy groups having about 1 to about 17 carbon atoms, such as difluoromethyloxy group, trifluoromethyloxy group, tetrafluoroethyloxy group, pentafluoroethyloxy group, and hexafluoroisopropyloxy group (2,2,2-trifluoro-1-trifluoromethylethoxy group), of which haloalkoxy groups having about 1 to about 10 carbon atoms are preferred. Of haloalkoxy groups, fluorinated alkyloxy groups are especially preferred. Exemplary other organic groups as X¹, X², X³, and X⁴ include alkylideneamino groups and cycloalkylideneamino groups. Leaving groups are preferred as X¹, X², X³, and X⁴. Incidentally, Z may be a leaving group other than a halogen atom or an —OR group.

In the compounds represented by Formulae (3a) to (3e), X¹, X², X³, and X⁴ can each be the same group as with Z, namely, they can each be a group selected from halogen atoms and —OR groups. A triazine derivative of Formula (3a), when X¹ and X² are each a group selected from halogen atoms and —OR groups, be an aromatic compound containing three structures represented by Formula (3) per one molecule. A pyrazine derivative represented by Formula (3b), a pyrimidine derivative represented by Formula (3c), and a pyridazine derivative represented by Formula (3d), when X³ is a group selected from halogen atoms and —OR groups, be each an aromatic compound containing two structures represented by Formula (3) per one molecule.

Specific examples of triazine derivatives represented by Formula (3a) include triazine derivatives having at least one halogen atom (of which chlorine atom is preferred) as substituent(s), such as 2-chloro-1,3,5-triazine, 2,4-dichloro-1,3,5-triazine, 2,4,6-trichloro-1,3,5-triazine (cyanuric chloride), 2-chloro-4,6-dihydroxy-1,3,5-triazine, 2-chloro-4,6-dinitro-1,3,5-triazine, 2-chloro-4-nitro-1,3,5-triazine, and 2-chloro-4,6-dioxymethyl-1,3,5-triazine; triazine derivatives having at least one haloalkoxy group as substituent(s), such as 2-hexafluoroisopropyloxy-1,3,5-triazine, 2,4-bis(hexafluoroisopropyloxy)-1,3,5-triazine, and 2,4,6-tris(hexafluoroisopropyloxy)-1,3,5-triazine; triazine derivatives having at least one cycloalkylideneaminooxy group as substituent(s), such as 2-cyclododecylideneaminooxy-1,3,5-triazine, 2,4-bis(cyclododecylideneaminooxy)-1,3,5-triazine, and 2,4,6-tris(cyclododecylideneaminooxy)-1,3,5-triazine; triazine derivatives having at least one halogen atom and at least one haloalkoxy group as substituents, such as 2-chloro-4,6-bis(hexafluoroisopropyloxy)-1,3,5-triazine and 2,4-dichloro-6-(hexafluoroisopropyloxy)-1,3,5-triazine; triazine derivatives having at least one halogen atom and at least one cycloalkylideneaminooxy group as substituents, such as 2-chloro-4-cyclododecylideneaminooxy-1,3,5-triazine; triazine derivatives having at least one cycloalkylideneaminooxy group and at least one haloalkoxy group as substituents, such as 2-cyclododecylideneaminooxy-4,6-bis(hexafluoroisopropyloxy)-1,3,5-triazine; and triazine derivatives having at least one halogen atom, at least one haloalkoxy group, and at least one cycloalkylideneamino group as substituents, such as 2-chloro-4-(hexafluoroisopropyloxy)-6-cyclododecylideneaminooxy-1,3,5-triazine.

Specific examples of pyrazine derivatives represented by Formula (3b) include pyrazine derivatives having at least one halogen atom as a substituent, such as 2-chloropyrazine, 2,3-dichloropyrazine, and 2-chloro-3,5-dinitropyrazine; pyrazine derivatives having at least one haloalkoxy group as a substituent, such as 2-(hexafluoroisopropyloxy)pyrazine; and pyrazine derivatives having at least one cycloalkylideneaminooxy group as a substituent, such as 2-cyclododecylideneaminooxypyrazine.

Specific examples of pyrimidine derivatives represented by Formula (3c) include pyrimidine derivatives having at least one halogen atom as a substituent, such as 2,4-dichloro-pyrimidine, 2,4,6-trichloropyrimidine, 4,6-dichloro-5-nitropyrimidine, and 2,4-dichloro-6-nitropyrimidine; pyrimidine derivatives having at least one haloalkoxy group as a substituent, such as 2,4-bis(hexafluoroisopropyloxy)pyrimidine; and pyrimidine derivatives having at least one cycloalkylideneaminooxy group as a substituent, such as 2,4-dicyclododecylideneaminooxypyrimidine.

Specific examples of pyridazine derivatives represented by Formula (3d) include pyridazine derivatives having at least one halogen atom as a substituent, such as 3-chloropyridazine and 3,6-dichloropyridazine; pyridazine derivatives having at least one haloalkoxy group as a substituent, such as 3-hexafluoroisopropyloxypyridazine; and pyridazine derivatives having at least one cycloalkylideneaminooxy group as a substituent, such as 3-cyclododecylideneaminooxypyridazine.

Specific examples of pyridine derivatives represented by Formula (3e) include pyridine derivatives having at least one halogen atom as a substituent, such as 2-chloro-3,5-dinitropyridine, 2,4,6-trichloropyridine, and 2-chloropyridine; pyridine derivatives having at least one haloalkoxy group as a substituent, such as 2-hexafluoroisopropyloxypyridine; and pyridine derivatives having at least one cycloalkylideneaminooxy group as a substituent, such as 2-cyclododecylideneaminooxypyridine.

Among them, triazine derivatives represented by Formula (3a) are preferably used, of which 2,4,6-trichloro-1,3,5-triazine, 2,4,6-tris(hexafluoroisopropyloxy)-1,3,5-triazine, and 2,4,6-tris(cyclododecylideneaminooxy)-1,3,5-triazine are more preferably used.

Exemplary aromatic compounds containing a structure represented by Formula (3) as a ring constituent further include compounds having a nitrogen-containing fused heterocyclic ring skeleton, such as quinoline, isoquinoline, quinazoline, quinoxaline, phthalazine, purine, pteridine, phenanthridine, and phenanthroline.

When the aromatic compound containing a structure represented by Formula (3) as a ring constituent is a compound having an —OR group as Z, the aromatic compound may be previously prepared before use in the reaction, but such aromatic compound having an —OR group as Z can also be formed within the reaction system, by incorporating a corresponding compound having a halogen atom as Z and a compound that generates an RO⁻ ion into the reaction system for the production of a lactam compound to allow a substitution reaction between the halogen atom and —OR group to proceed in the reaction system. Though not especially limited, the compound that generates an RO⁻ ion is often the oxime compound used as a raw material. Specifically, embodiments of the present invention in which Z is an —OR group include an embodiment in which an aromatic compound containing, as a ring constituent, a structure represented by Formula (3) wherein Z is a halogen atom is used, and this aromatic compound reacts with an oxime compound as the raw material to give an aromatic compound having a group corresponding to the oxime compound, except for removing hydrogen atom therefrom (e.g., cycloalkylideneaminooxy group) as a substituent.

Of aromatic compounds A, exemplary aromatic compounds other than the aromatic compounds containing a structure represented by Formula (3) as a ring constituent include benzene derivatives such as 4-chloro-3,5-dinitrobenzonitrile and picryl chloride.

Each of different aromatic compounds A may be used alone or in combination. The amount of aromatic compounds A in a rearrangement reaction of a cyclic oxime compound is for example, from about 0.0001 to about 1 mole, preferably from about 0.0005 to about 0.5 mole, and more preferably from about 0.001 to about 0.2 mole, per 1 mole of the cyclic oxime compound represented by Formula (1).

[Promoter]

A promoter (co-catalyst) may be used in combination with the aromatic compound A in the method according to the present invention. Exemplary promoters include acids such as Lewis acids and Broensted acids. The Lewis acids can be common Lewis acids. Exemplary Broensted acids include inorganic acids such as hydrochloric acid; and organic acids including carboxylic acids such as monochloroacetic acid, dichloroacetic acid, trichloroacetic acid, and trifluoroacetic acid, and sulfonic acids such as methanesulfonic acid, trifluoromethanesulfonic acid, and p-toluenesulfonic acid. Among them, sulfonic acids are especially preferred. Each of different promoters may be used alone or in combination.

The amount of promoters, if used, is typically from about 0.0001 to about 1 mole, preferably from about 0.0005 to about 0.5 mole, and more preferably from about 0.001 to about 0.2 mole, per 1 mole of the cyclic oxime compound represented by Formula (1).

[Nonpolar Solvent B]

Exemplary nonpolar solvents B are organic solvents that will be separated from water. Examples thereof include hydrocarbons including aliphatic hydrocarbons such as pentane, hexane, heptane, octane, decane, and dodecane; alicyclic hydrocarbons such as cyclopentane, cyclohexane, cyclooctane, cyclodecane, cyclododecane, and cyclopentadecane; aromatic hydrocarbons such as benzene, toluene, xylenes, ethylbenzene, and mesitylene; chain ethers such as dipropyl ether, diisopropyl ether, dibutyl ether, and dihexyl ether; and halogenated hydrocarbons such as methylene chloride, dichloroethane, chloroform, carbon tetrachloride, chlorobenzene, and trifluoromethylbenzene. Each of different nonpolar solvents B may be used alone or in combination, Among these solvents, hydrocarbons are preferred as nonpolar solvents.

The nonpolar solvent B is preferably a cycloalkane corresponding the cyclic oxime compound represented by Formula (1). The reasons for this are as follows. The cyclic oxime compound having 8 to 15 carbon atoms for use as the raw material in the present invention is prepared by a process of oxidizing a cycloalkane having 8 to 15 carbon atoms into a corresponding cyclic ketone and allowing hydroxylamine to react with the cyclic ketone; or a process of allowing a nitrous ester or nitrite (salt of nitrous acid) to react with a cycloalkane having 8 to 15 carbon atoms. In a rearrangement reaction of a cyclic oxime compound represented by Formula (1) according to the present invention, when a cycloalkane corresponding to the cyclic oxime compound represented by Formula (1) is used as a solvent, residual cycloalkane which has been unreacted in a precedent step can be used as the solvent, and this eliminates the need of separation between a reaction product and the unreacted raw material (cycloalkane) in a precedent step. The reaction mixture obtained in the precedent step can therefore be fed to this step without any treatment or with a simple treatment such as separation of the catalyst through extraction, and this technique is very advantageous in respect of both energy and process. The oxidation reaction of the cycloalkane, the oximation reaction of the cyclic ketone, and the reaction between the cycloalkane and the nitrous ester or nitrite can be carried out according to known procedures.

The amount of nonpolar solvents B in the rearrangement reaction of the cyclic oxime compound is, for example, from about 0.1 to about 50 times by weight, preferably from about 0.5 to about 20 times by weight, and more preferably from about 1 to about 10 times by weight, the weight of the cyclic oxime compound represented by Formula (1).

[Cyclic Oxime Compound]

Exemplary cyclic oxime compounds represented by Formula (1) include cyclooctanone oxime, cyclononanone oxime, cyclodecanone oxime, cycloundecanone oxime, cyclododecanone oxime, cyclotridecanone oxime, cyclotetradecanone oxime, and cyclopentadecanone oxime. Among them, cyclododecanone oxime is especially preferably used.

[Rearrangement Reaction]

The reaction temperature in the rearrangement reaction of the cyclic oxime compound can be set according typically to the types of the cyclic oxime compound, catalyst, and solvent to be used, is not especially limited, but is generally from about 0° C. to about 250° C., preferably from about 25° C. to about 150° C., and more preferably from about 40° C. to about 120° C. The reaction may be carried out in an atmosphere of an inert gas such as nitrogen or argon gas, or can be carried out in an atmosphere of air or oxygen. The reaction herein is especially preferably carried out in an air atmosphere under reflux conditions. The reaction can be carried out under reduced pressure, under normal atmospheric pressure, or under a pressure (under a load), according to any system such as a batch system, semi-batch system, or continuous system (e.g., multistage continuous circulation system).

As a result of the reaction, the raw material cyclic oxime compound gives a corresponding lactam compound which is represented by Formula (2) and contains one more member than the cyclic oxime compound. For example, cyclododecanone oxime gives laurolactam.

After the completion of the reaction, a reaction product can be separated and purified, for example, through a separation procedure such as filtration, concentration, distillation, extraction, crystallization, recrystallization, adsorption, or column chromatography, or any combination of them. Typically, the resulting lactam compound can be separated from the catalyst aromatic compound A by subjecting them to extraction using an organic solvent and water to transfer the lactam compound into an organic layer and the aromatic compound A into an aqueous layer, respectively.

[Production of Cyclic Oxime Compound]

The cyclic oxime compound represented by Formula (1) is very advantageously produced, for example, by the process mentioned below. According to the process, the cyclic oxime compound can be efficiently produced in a simple manner under mild conditions, and it is possible that a reaction for the synthesis of the cyclic oxime compound and a reaction for the production of a corresponding lactam compound through the rearrangement of the cyclic oxime compound can be conducted in one step (in one pot) without need of an extra intermediate step for separating and purifying the cyclic oxime compound.

Specifically, the cyclic oxime compound represented by Formula (1) is preferably produced by allowing a cycloalkane corresponding to the cyclic oxime compound represented by Formula (1) to react with a nitrous ester or nitrite in the presence of a nitrogen-containing cyclic compound containing, as a ring constituent, a skeleton represented by following Formula (6):

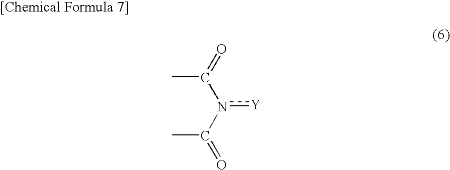

wherein Y represents an oxygen atom or an —OR′ group, where R′ represents a hydrogen atom or a hydroxyl-protecting group.

Specific examples of the nitrogen-containing cyclic compound containing a skeleton represented by Formula (6) as a ring constituent for use herein include N-hydroxyimide compounds derived from aliphatic polycarboxylic acid anhydrides (cyclic anhydrides) or aromatic polycarboxylic acid anhydrides (cyclic anhydrides), such as N-hydroxysuccinimide, N-hydroxyphthalimide, N,N′-dihydroxypyromellitic diimide, N-hydroxyglutarimide, N-hydroxy-1,8-naphthalenedicarboximide, and N,N′-dihydroxy-1,8,4,5-naphthalenetetracarboxylic diimide; and compounds obtained by introducing a protecting group (e.g., an acyl group such as acetyl group) into a hydroxyl group of the N-hydroxy imide compounds.

Exemplary cycloalkanes include cyclooctane, cyclononane, cyclodecane, cycloundecane, cyclododecane, cyclotridecane, cyclotetradecane, and cyclopentadecane.

Exemplary nitrous esters include alkyl nitrites such as methyl nitrite, ethyl nitrite, propyl nitrite, isopropyl nitrite, butyl nitrite, isobutyl nitrite, t-butyl nitrite, amyl nitrite, isoamyl nitrite, t-amyl nitrite, and hexyl nitrite; aryl nitrites such as phenyl nitrite; and aralkyl nitrites such as benzyl nitrite. Preferred exemplary nitrous esters include alkyl nitrites including alkyl nitrites whose alkyl moiety having 1 to 6 carbon atoms. Exemplary nitrites (salts of nitrous acid) include ammonium nitrite; nitrites of alkaline earth metals, such as lithium nitrite, sodium nitrite, potassium nitrite, and barium nitrite; and nitrites of other metals, such as zinc nitrite.

The proportion between the cycloalkane and the nitrous ester or nitrite can be selected as appropriate according typically to the types and combination of the two compounds. Typically, the cycloalkane may be used in an amount substantially equivalent or in excess (e.g., from about 1.1 to about 50 times by equivalent or more, and preferably from about 3 to about 30 times by equivalent) to the nitrous ester or nitrite. Contrarily, the nitrous ester or nitrite may be used in excess to the cycloalkane.

The reaction between the cycloalkane and the nitrous ester or nitrite is carried out in the presence of, or in the absence of, a solvent. The solvent is not especially limited, and the solvents listed in the rearrangement reaction of the oxime compound can also be used herein. Among such solvents, the cycloalkane itself is preferably used as the solvent, as described above. In this case, unreacted cycloalkane can be used as the solvent in the step of lactamization according to the present invention, and whereby a target lactam compound can be produced very efficiently. The reaction temperature and other conditions are not especially limited, and the reaction herein can be carried out typically under the same or similar conditions to those in the rearrangement reaction of the oxime compound. Typically, the reaction temperature is from about 0° C. to about 250° C., preferably from about 25° C. to about 150° C., and more preferably from about 40° C. to about 120° C. The reaction may be carried out in an atmosphere of an inert gas such as nitrogen or argon gas, but it can be carried out in an air atmosphere or oxygen atmosphere typically in the case of target products of some types. The reaction can be carried out under reduced pressure, under normal atmospheric pressure, or under a pressure (under a load), according to a common system or procedure such as a batch system, semi-batch system, or continuous system (e.g., multistage continuous circulation system). The yield is significantly improved when the reaction is carried out under reduced pressure, especially under such a reduced pressure that nitrogen oxide gases (particularly NO₂) produced as by-products as a result of the reaction can be removed from the system [e.g., from about 30 to about 700 mmHg (from about 3.99 to about 93.1 kPa)]. This is probably because nitrogen oxide gases such as NO₂ will inhibit the reaction.

It is considered that the reaction between a cycloalkane and a nitrous ester or nitrite initially gives a nitroso compound, and this compound is rearranged to give an oxime compound. For example, it is considered that the reaction between cyclododecane and a nitrous ester or nitrite initially gives nitrosocyclododecane, and this is rearranged to give cyclododecanone oxime. Though varying from type to type, a nitroso compound of some type may be in reversible equilibrium with a corresponding dimer (di-N-oxide compound in which two molecules of the nitroso compound are bonded through their nitrogen atoms) and the equilibrium may lie to the dimer. When the reaction is carried out over a long period of time, the nitroso compound and a dimer thereof can be in a trace amount, at most in a yield of less than 1%.

In a preferred embodiment, the reaction between the cycloalkane and the nitrous ester or nitrite is carried out by consecutively or continuously adding the nitrous ester or nitrite to the reaction system. According to this embodiment, side reactions particularly in nitrosation stage can be suppressed, and thereby the nitroso compound (or a dimer thereof) can be produced with a high selectivity, as compared to a process of adding the nitrous ester or nitrite at once, Thus, the oxime compound can be obtained in a high yield typically through the subsequent rearrangement reaction.

In another embodiment, for producing an oxime compound in a good yield, reactions are allowed to proceed stepwisely by providing the step of reacting a cycloalkane with a nitrous ester or nitrite to give a nitroso compound or a dimer thereof and the step of converting the resulting nitroso compound or dimer thereof into the oxime compound. In this embodiment, the total reaction time can be significantly shortened by adding an additive to the reaction system or carrying out heating in the subsequent conversion step (rearrangement step of the nitroso compound). The subsequent rearrangement step may use another solvent than the solvent used in the precedent nitrosation step. In this embodiment, the precedent nitrosation step is preferably carried out under reduced pressure, because the yield is significantly improved for the same reason as above.

Though not especially limited, as long as being capable of inducing the rearrangement from a nitroso form to an oxime form, the additive is preferably selected typically from acids and bases. Exemplary acids herein include sulfonic acids such as methanesulfonic acid, trifluoromethanesulfonic acid, benzenesulfonic acid, and p-toluenesulfonic acid; mineral acids such as sulfuric acid, nitric acid, hydrochloric acid, phosphoric acid, boric acid, and fuming sulfuric acid; Lewis acids such as aluminum chloride, zinc chloride, and scandium triflate; solid acids such as silica, alumina, and zeolite; complex acids including polyacids such as phosphomolybdic acid, phosphotungstic acid, silicomolybdic acid, and silicotungstic acid; and strongly acidic cation-exchange resins. Exemplary bases include organic bases including tertiary amines such as triethylamine, nitrogen-containing heterocyclic compounds such as pyridine, as well as sodium acetate, and sodium methoxide; inorganic bases such as sodium carbonate, sodium hydrogen carbonate, sodium hydroxide, and potassium hydroxide; and solid bases such as magnesium oxide, hydrotalcite, and hydroxyapatite. The additives may be added at once or in two or more installments. The amount of additives is, for example, from about 0.01 to about 100 parts by weight, preferably from about 0.1 to about 50 parts by weight, and more preferably from about 0.3 to about 30 parts by weight, per 100 parts by weight of the cycloalkane. A rearrangement reaction using additives may be carried out at a temperature of, for example, from about 40° C. to about 120° C., and preferably from about 50° C. to about 100° C. for a duration of, for example, from about 5 to about 180 minutes, and preferably from about 10 to about 120 minutes. A rearrangement reaction with heating may be carried out at a heating temperature of, for example, from about 120° C. to about 250° C., and preferably from about 150° C. to about 200° C. for a reaction time of, for example, from about 0.5 to about 120 minutes, and preferably from about 2 to about 90 minutes.

In the production of an oxime compound, it is possible to produce a corresponding lactam from a cycloalkane in one step by simultaneously adding the aromatic compound A in addition to the cycloalkane, a nitrous ester or nitrite, and a nitrogen-containing cyclic compound having a skeleton represented by Formula (6) as a ring constituent, and carrying out a reaction. In another possible process, a reaction between a cycloalkane and a nitrous ester or nitrite is carried out in the presence of a nitrogen-containing cyclic compound containing a skeleton represented by Formula (6) as a ring constituent to give an oxime compound and the aromatic compound A is added to the oxime compound, followed by carrying out the rearrangement reaction of the oxime compound. In these methods, an operation such as distilling off of the solvent, concentration, or exchange of the solvent may be carried out in an appropriate stage. The production of the oxime compound may be conducted stepwisely, as described above.

According to the methods of the present invention, lactams can be produced in a high yield in a simple manner without causing large amounts of by-products. Additionally, high-purity lactams can be produced in a simple manner, because the catalyst for use in the present invention is easily separable from the produced lactams. Furthermore, reaction products after the completion of the reaction can be separated from the used catalyst according to a simple procedure such as an extraction operation using water, because a nonpolar solvent is used. It is possible to carry out the step of producing an oxime from a cycloalkane and the step of producing a lactam from the oxime compound as one step or in one pot. The methods according to the present invention are therefore very useful as methods for industrially producing lactam compounds having 8 to 15 carbon atoms (especially ω-laurolactam).

The resulting lactams are usable typically as raw materials for pharmaceutical drugs, agricultural chemicals, dyestuffs, solvents, and explosives; and as raw materials for polyamides (nylons).

### EXAMPLES

The present invention will be illustrated in further detail with reference to several examples below, which, however, are by no means intended to limit the scope of the present invention.

### Example 1

In a reactor were placed cyclododecanone oxime (10 mmol), 2,4,6-trichloro-1,3,5-triazine (2 percent by mole), and toluene (8 mL), followed by stirring at 80° C. for 2 hours. A gas chromatographic analysis was conducted after the reaction to find that laurolactam was produced in a yield of 95%.

### Example 2

In a reactor were placed cyclododecanone oxime (10 mmol), 2,4,6-trichloro-1,3,5-triazine (2 percent by mole), and cyclododecane (8 g), followed by stirring at 80° C. for 2 hours. A gas chromatographic analysis was conducted after the reaction to find that laurolactam was produced in a yield of 96%.

### Example 3

In a reactor were placed cyclododecanone oxime (10 mmol), 2,4,6-trichloro-1,3,5-triazine (2 percent by mole), and toluene (8 mL), followed by stirring at 90° C. for 2 hours. A gas chromatographic analysis was conducted after the reaction to find that laurolactam was produced in a yield of 87%.

### Example 4

In a reactor were placed cyclododecanone oxime (10 mmol), 2,4,6-trichloro-1,3,5-triazine (2 percent by mole), and isopropylcyclohexane (8 mL), followed by stirring at 80° C. for 2 hours. A gas chromatographic analysis was conducted after the reaction to find that laurolactam was produced in a yield of 95%.

### Example 5

In a reactor were placed cyclooctanone oxime (10 mmol), 2,4,6-trichloro-1,3,5-triazine (2 percent by mole), and toluene (8 mL), followed by stirring at 80° C. for 2 hours. A gas chromatographic analysis was conducted after the reaction to find that a corresponding lactam was produced in a yield of 65%.

### Example 6

In a reactor were placed cyclopentadecanone oxime (10 mmol), 2,4,6-trichloro-1,3,5-triazine (2 percent by mole), and toluene (8 mL), followed by stirring at 80° C. for 2 hours. A gas chromatographic analysis was conducted after the reaction to find that a corresponding lactam was produced in a yield of 72%.

### Example 7

In a reactor were placed cyclododecanone oxime (10 mmol), 2,4,6-trichloro-1,3,5-triazine (2 percent by mole), and toluene (8 mL), followed by stirring at 70° C. for 2 hours. A gas chromatographic analysis was conducted after the reaction to find that laurolactam was produced in a yield of 98%.

### Example 8

In a reactor were placed cyclododecanone oxime (10 mmol), 2,4,6-tris(hexafluoroisopropyloxy)-1,3,5-triazine [i.e., 2,4,6-tris(2,2,2-trifluoro-1-trifluoromethylethoxy)1,3,5-triazine] (0.5 percent by mole), and toluene (8 mL), followed by stirring under reflux conditions for 2 hours. A gas chromatographic analysis was conducted after the reaction to find that laurolactam was produced in a yield of 80%.

### Example 9

In a reactor were placed cyclododecanone oxime (10 mmol), 2,4,6-tris(cyclododecylideneaminooxy)-1,3,5-triazine [i.e., O-4,6-bis(cyclododecylideneaminooxy)-1,3,5-triazin-2-ylcyclododecanone oxime] (0.5 percent by mole), and toluene mL), followed by stirring under reflux conditions for 2 hours. A gas chromatographic analysis was conducted after the reaction to find that laurolactam was produced in a yield of 70%.

### Example 10

In a reactor were placed cyclododecanone oxime (10 mmol), 2,4,6-tris(hexafluoroisopropyloxy)-1,3,5-triazine [i.e., 2,4,6-tris(2,2,2-trifluoro-1-trifluoromethylethoxy)1,3,5-triazine] (0.5 percent by mole), p-toluenesulfonic acid (5 percent by mole), and cyclododecane (8 g), followed by stirring under reflux conditions for 2 hours. A gas chromatographic analysis was conducted after the reaction to find that laurolactam was produced in a yield of 90%.

### Example 11

In a reactor were placed cyclododecanone oxime (10 mmol), 2,4,6-tris(cyclododecylideneaminooxy)-1,3,5-triazine [i.e., O-4,6-bis(cyclododecylideneaminooxy)-1,3,5-triazin-2-ylcyclododecanone oxime] (0.5 percent by mole), p-toluenesulfonic acid (5 percent by mole), and cyclododecane (8 g), followed by stirring under reflux conditions for 2 hours. A gas chromatographic analysis was conducted after the reaction to find that laurolactam was produced in a yield of 92%.

### Comparative Example 1

In a reactor were placed cyclohexanone oxime (10 mmol), 2,4,6-trichloro-1,3,5-triazine (2 percent by mole), and toluene (2 mL), followed by stirring at 80° C. for 2 hours. A gas chromatographic analysis was conducted after the reaction to find that caprolactam was produced in a yield of 5%.

### Example 12

In a reactor were placed cyclododecanone oxime (10 mmol), 4-chloro-3,5-dinitrobenzonitrile (0.5 percent by mole), and cyclododecane (8 g), followed by stirring at 80° C. for 2 hours. A gas chromatographic analysis was conducted after the reaction to find that laurolactam was produced in a yield of 74%.

### Example 13

In a reactor were placed cyclododecanone oxime (10 mmol), 4-chloro-3,5-dinitrobenzonitrile (0.5 percent by mole), and toluene (8 mL), followed by stirring at 80° C. for 2 hours. A gas chromatographic analysis was conducted after the reaction to find that laurolactam was produced in a yield of 76%.

### Comparative Example 2

In a reactor were placed cyclododecanone oxime (10 mmol), and cyclododecane (8 g), followed by stirring at 80° C. for 2 hours. A gas chromatographic analysis was conducted after the reaction to find that no reaction had proceeded.

### INDUSTRIAL APPLICABILITY

According to the present invention, lactam compounds having 8 to 15 carbon atoms can be produced through rearrangement reactions of cyclic oximes without causing large amounts of by-products such as ammonium sulfate, and this solves problems occurring in known methods for producing lactam compounds, such as removal and disposal of by-products. Additionally, reaction products after the completion of the reaction can be easily separated from the used catalyst, because a nonpolar solvent is used. The resulting lactam compounds are useful typically as raw materials for pharmaceutical drugs, agricultural chemicals, dyestuffs, solvents, and explosives, and as raw materials for polyamides (nylons).

1. A method for producing a lactam compound, the method comprising the step of carrying out rearrangement of a cyclic oxime compound in a nonpolar solvent B in the presence of an aromatic compound A to give the lactam compound, the aromatic compound A having a leaving group bonded to a carbon atom constituting its aromatic ring, the aromatic compound A containing, as an atom constituting its aromatic ring, a heteroatom, or a carbon atom bonded with an electron-withdrawing group, the cyclic oxime compound being represented by following Formula (1): 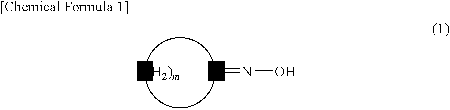
wherein “m” denotes an integer of from 7 to 14,
and the lactam compound being represented by following Formula (2): 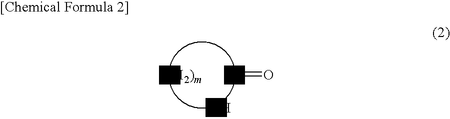
wherein “m” is as defined above.

2. The method for producing a lactam compound, according to claim 1, wherein the aromatic compound A is an aromatic compound containing, as a constituent of the aromatic ring, a structure represented by following Formula (3): 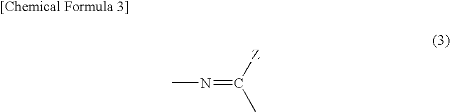
wherein Z represents a halogen atom or an —OR group, where R represents an organic group.

3. The method for producing a lactam compound, according to claim 2, wherein Z in Formula (3) is chlorine atom.

4. The method for producing a lactam compound, according to claim 2, wherein R in Formula (3) is a fluorine-containing branched-chain aliphatic group represented by following Formula (4): 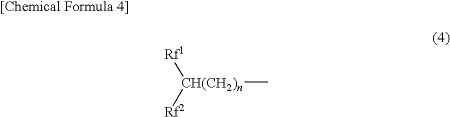
wherein Rf¹ and Rf² are the same as or different from each other and each represent a perfluoroalkyl group having 1 to 8 carbon atoms; and “n” denotes an integer of from 0 to 8.

5. The method for producing a lactam compound, according to claim 2, wherein R in Formula (3) is a group corresponding to the cyclic oxime compound represented by Formula (1) and being represented by following Formula (5): 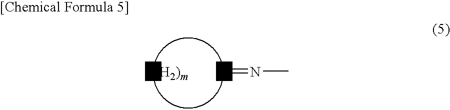
wherein “m” denotes an integer of from 7 to 14.

6. The method for producing a lactam compound, according to claim 1, wherein the nonpolar solvent B is a hydrocarbon.

7. The method for producing a lactam compound, according to claim 1, wherein the nonpolar solvent B is a cycloalkane corresponding to the cyclic oxime compound represented by Formula (1).

8. The method for producing a lactam compound, according to claim 1, further comprising using an acid as a promoter.
